(19) United States
(12) Patent Application Publication
(10) Pub. No.: US 2017/0245956 A1
(43) Pub. Date: Aug. 31, 2017

(54) EYE DROP DISPENSER STORAGE CASE

(71) Applicant: Magida Abu-moustafa, Boca Raton, FL (US)

(72) Inventor: Magida Abu-moustafa, Boca Raton, FL (US)

(21) Appl. No.: 15/056,644

(22) Filed: Feb. 29, 2016

(51) Int. Cl. A61B 50/30; B65D 25/10

(52) U.S. Cl. CPC A61B 50/30; B65D 25/10; A61B 2050/0016

(57) ABSTRACT

A temperature controlled case to store and manage the time-sensitive management of personal eye drops or similar medicine. The case may include a first row of dispenser holding compartments for holding dispensers for the drops ‘to be taken’ and a second row of dispenser holding compartments for holding the dispensers for the drops ‘already taken’. A clock timer to manage the required time intervals during which the drops must be taken may also be included. The case may include side compartments for holding other items (e.g., cotton balls, cotton swabs) that are typically required when a person uses eye drops.

### FIELD OF THE INVENTION

Embodiments disclosed herein relate to a storage case for medicinal and other dispensers and, in particular, to a storage case for eye drop dispensers.

### BACKGROUND

There are many known eye maladies, such as e.g., dry eyes and other serious eye conditions such as glaucoma. Glaucoma, for example, occurs when a build-up of fluid in the eye creates pressure, damaging the optic nerve. The treatment for glaucoma includes taking multiple eye drops, in a specific sequence, within specific time intervals, at specific times throughout the day. Often times the effected individual finds it challenging to balance this important routine and keep track of which drops were already taken. It is even more challenging for the individual to remember how much time elapsed since the drops were taken. What initially sounds like a relatively simple process can easily become very confusing, not to mention dangerous if not done correctly.

Eye drops for glaucoma, for example, are also required to be maintained at a desired temperature. Thus, the challenges experienced by the effected individual become even more difficult when she is away from home and forced to carry one or more eye drop dispensers in a cooler or bags to maintain the drops at their proper temperature.

Currently, there are no solutions to these very important problems. Accordingly, there is a need and desire for a multi-utility storage case for eye drop dispensers that provides storage, temperature control and organizational means to help with the administration of their contents.

### SUMMARY

The embodiments disclosed herein provide a temperature controlled case to store dropper dispensers for e.g., eye drops or other similar medicine. The embodiments disclose herein further provide for and manage the time-sensitive administration of the eye drops using unique compartments and a timer built into or formed as part of the case. In one embodiment, the case may include a first row of dispenser holding compartments for holding dispensers for the drops ‘to be taken’ and a second row of dispenser holding compartments for holding the dispensers for the drops ‘already taken’. Between the two rows in the center of the case is a clock timer to manage the required time intervals during which the drops must be taken. In one embodiment, the case may include side compartments for holding other items (e.g., cotton balls, cotton swabs, etc.) that are typically required when a person uses eye drops. Moreover, in one embodiment, the case may include a closure mechanism and is sized to fit into a purse, briefcase, or large pocket.

### BRIEF DESCRIPTION OF THE DRAWING

FIG. 1 illustrates a top view of a storage case constructed in accordance with an embodiment disclosed herein.

FIG. 2 illustrates a side view of the storage case constructed in accordance with an embodiment disclosed herein.

### DETAILED DESCRIPTION

The disclosed principles are described as being used to store eye drop dispensers and to maintain the contents of the dispensers (i.e., eye drops) at an appropriate temperature. It should be appreciated, however, that the disclosed principles could provide the same functionality to other types of dispensers, bottles, etc. and their contents should there be a need to do so.

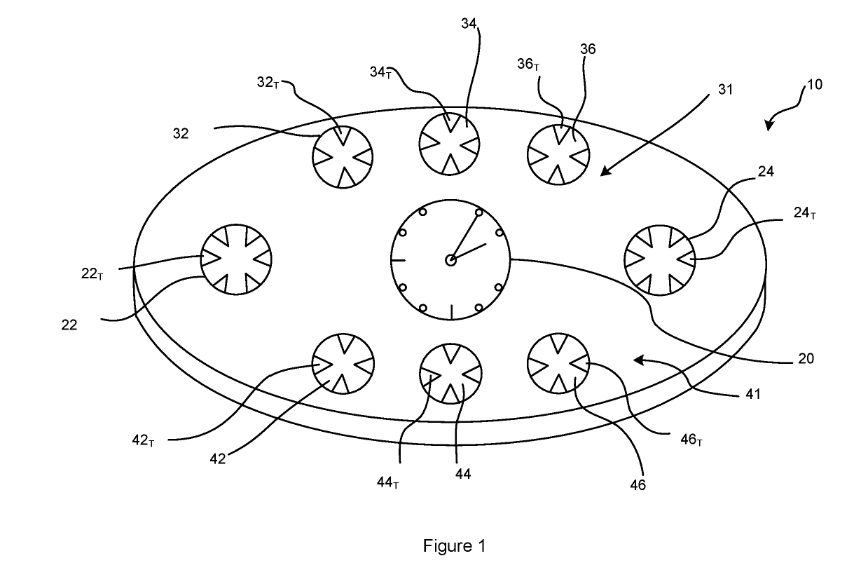

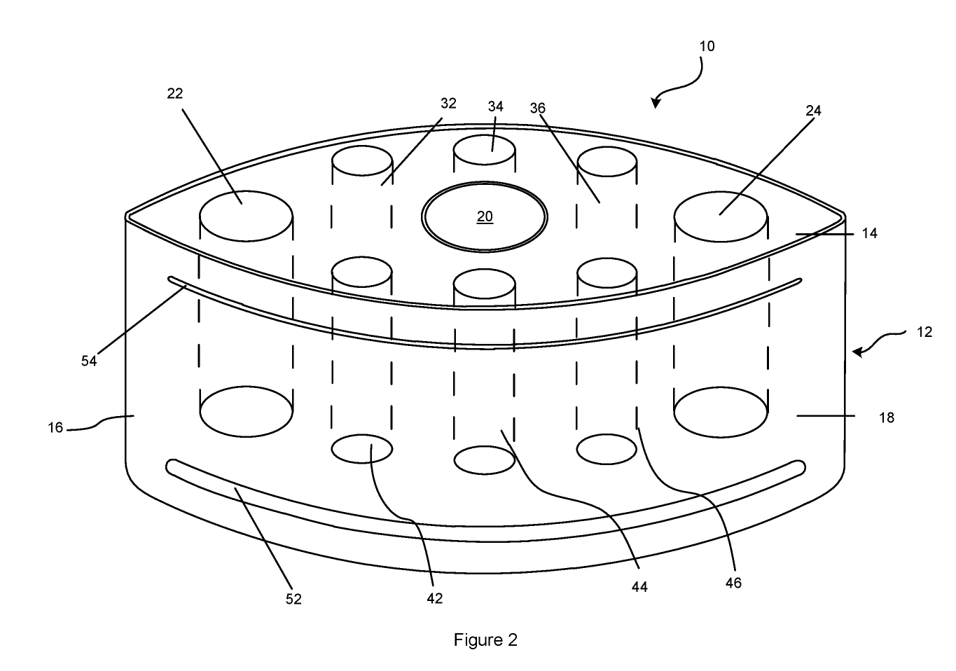

FIG. 1 illustrates a top view of a storage case 10 constructed in accordance with an embodiment disclosed herein while FIG. 2 illustrates a side view of the storage case 10. In one embodiment, the case 10 may include a cotton-lined housing 12 comprising a top portion 14 connected to sidewall portions 16. The sidewall portions 16 are further connected to a bottom portion (not shown). In one embodiment, the dimensions of the housing 12 (and thus, the case 10) are approximately four inches in height by eight inches in length. The case 10 is sized such that it can easily fit into e.g., a purse, briefcase, or large pocket. Because the illustrated embodiment is preferably used to store eye drop dispensers, the housing 12, and thus the case 10, is formed in the shape of a human eye (i.e., an oval). It should be appreciated, however, that the case 10 can have other shapes (e.g., circular, square-shaped, rectangular-shaped, etc.) and dimensions if desired and that the principles disclosed herein should not be limited solely to the use of an eye-shaped case 10.

The illustrated embodiment may include a zipper 54 to seal the top portion 14 of the housing 12 to the sidewall portions 16, enclosing an internal portion 18 of the case 10. It should be appreciated that other fastening mechanisms (e.g., hook and loop fasteners (i.e., VELCRO), buttons, etc.) could be used in place of the zipper 54.

In the illustrated embodiment, the internal portion 18 may be made from a sponge-like material and includes a first row of dispenser holding compartments 32, 34, 36 and a second row of dispenser holding compartments 42, 44, 46 formed therein. In the illustrated embodiment, each row 31, 41 respectively comprises three dispenser holding compartments 32, 34, 36, 42, 44, 46. It should be appreciated that more or less dispenser holding compartment 32, 34, 36, 42, 44, 46 can be included in the case 10 if desired.

Each dispenser holding compartment 32, 34, 36, 42, 44, 46 may include tentacle-like grippers 32_{T}, 34_{T}, 36_{T}, 42_{T}, 44_{T}, 46_{T} to secure dispensers in an upright manner while also compensating for the varying sizes of e.g., eye drop dispensers available in today's market. It should be appreciated that the materials for the internal portion 18 and the grippers 32_{T}, 34_{T}, 36_{T}, 42_{T}, 44_{T}, 46_{T} may be any material suitable for maintaining dispensers. In addition, each dispenser holding compartment 32, 34, 36, 42, 44, 46 may have a diameter of approximately three-quarters of an inch and a depth of about two and a half inches. It should be appreciated, however, that the dispenser holding compartments 32, 34, 36, 42, 44, 46 can have other shapes and dimensions if desired and that the principles disclosed herein should not be limited solely to those disclosed herein.

While the dispenser holding compartments 32, 34, 36, 42, 44, 46 may be used in any manner the holder of the case 10 desires, it is intended that one row 31 (or 41) be used to store the dispensers for the drops ‘to be taken’ while the other row 41 (or 31) be used to store the dispensers for the drops ‘already taken’. To that end, the top portion 14 of the housing 12 can include markings or other indicia suggesting that e.g., the first row 31 be used to store the dispensers for the drops ‘to be taken’ and markings or other indicia suggesting that the second row 41 be used to store the dispensers for the drops ‘already taken’. Likewise, the top portion 14 of the housing 12 can include markings or other indicia suggesting that e.g., the second row 41 be used to store the dispensers for the drops ‘to be taken’ and markings or other indicia suggesting that the first row 31 be used to store the dispensers for the drops ‘already taken’. Thus, in an example use of the case 10, the first row 31 will accommodate up to three dispensers of eye drops to be taken and the second row 41, initially empty, will accommodate the same three bottles once their respective dose has been taken.

In addition, the illustrated embodiment includes, between the two rows 31, 41 in the center of the case 10 (e.g., the pupil of the eye), a timer 20 to manage the required time intervals during which the drops associated with the dispensers maintained in the case 10 must be taken. The timer 20 may be a standard timer that is reset by the individual each time a dispenser is used. In addition to, or alternatively, the timer 20 may be programmed to include a timer value for each dispenser stored within the case 10 (thus, alleviating the need for the individual to reset the timer 20 until all drops have been taken). In one embodiment, the timer 20 may have a diameter of approximately one and a half inches. It should be appreciated, however, that the timer 20 can have other shapes and dimensions if desired and that the principles disclosed herein should not be limited solely to those disclosed herein.

As can be seen, in the illustrated embodiment, the internal portion 18 further includes two side compartments 22, 24 formed therein. While the side compartments 22, 24 may be used in any manner the holder of the case 10 desires, it is intended that the side compartments 22, 24 hold items such as e.g., cotton balls and/or cotton swabs (i.e., Q-tips) that are typically used to wipe residue away from the eyes/face after application of viscous drops (as is often required when a person uses eye drops). It should be appreciated that more or less side compartments 22, 24 can be included in the case 10 if desired. Each side compartment 22, 24 may include tentacle-like grippers 22_{T}, 24_{T} to secure contents stored therein in a safe manner The grippers 22_{T}, 24_{T} may be any material suitable for maintaining items within the compartments 22, 24. In addition, each side compartment 22, 24 may have a diameter of at least approximately three-quarters of an inch and a depth of about two and a half inches. It should be appreciated, however, that the side compartments 22, 24 can have other shapes and dimensions if desired and that the principles disclosed herein should not be limited solely to those disclosed herein

An internal pouch 52 is formed through a sidewall portion 16 and into the internal portion 18 beneath the storage compartments 22, 24, 32, 34, 36, 42, 44, 46. The pouch may be approximately one-half inch deep and most of the length of the housing 12 and is intended to store a gel ice pack or other cooling mechanisms to keep the eye drops cool during transport. Additionally, or alternatively, the pouch 52 may store a heating element should the dispensers within the compartments 22, 24, 32, 34, 36, 42, 44, 46 require heat to maintain their contents at a desired temperature. The pouch 52 may comprise any material suitable for storing heating or cooling devices. The pouch 52, therefore, may be considered to be a temperature controlling compartment. The pouch 52 is accessed via a slit opening at the base of the case 10 and may be secured by a zipper, hook and loop fasteners (i.e., VELCRO) or other fastening mechanisms.

The foregoing examples are provided merely for the purpose of explanation and are in no way to be construed as limiting. While reference to various embodiments is made, the words used herein are words of description and illustration, rather than words of limitation. Further, although reference to particular means, materials, and embodiments are shown, there is no limitation to the particulars disclosed herein. Rather, the embodiments extend to all functionally equivalent structures, methods, and uses, such as are within the scope of the appended claims.

Additionally, the purpose of the Abstract is to enable the patent office and the public generally, and especially the scientists, engineers and practitioners in the art who are not familiar with patent or legal terms or phraseology, to determine quickly from a cursory inspection the nature of the technical disclosure of the application. The Abstract is not intended to be limiting as to the scope of the present inventions in any way.

What is claimed is:

1. A storage case comprising:
a housing having a portion with first and second rows of dispenser holding compartments formed therein, the portion of the housing having a temperature controlling compartment adapted to receive a heating or cooling device for heating or cooling contents within the dispenser holding compartments.

2. The storage case of claim 1, wherein the temperature controlling compartment comprises a fastening mechanism.

3. The storage case of claim 1, further comprising a timer formed between the first and second rows of dispenser holding compartments.

4. The storage case of claim 3, further comprising a plurality of side compartments formed within the portion of the housing.

5. The storage case of claim 3, further comprising a closing mechanism for maintaining a top portion of the housing secured to sidewall portions of the housing.

6. The storage case of claim 3, wherein the housing is oval shaped.

7. The storage case of claim 3, wherein each of first and second rows of dispenser holding compartments comprises three dispenser holding compartments.

8. The storage case of claim 7, wherein each dispenser holding compartment comprises grippers formed therein.

9. A case for storing a plurality of eye drop dispensers, the case comprising:
a housing having a portion with first and second rows of dispenser holding compartments formed therein, the portion of the housing having a temperature controlling compartment adapted to receive a heating or cooling device for heating or cooling contents within the dispenser holding compartments, the portion of the housing comprising a timer formed between the first and second rows of dispenser holding compartments.

10. The case of claim 9, wherein the temperature controlling compartment comprises a fastening mechanism selected from the group of a hook and loop fastener and a zipper.

11. The case of claim 9, further comprising two side compartments formed within the portion of the housing.

12. The case of claim 11, further comprising a closing mechanism for maintaining a top portion of the housing secured to sidewall portions of the housing.

13. The case of claim 11, wherein the housing is oval shaped.

14. The case of claim 11, wherein each of first and second rows of dispenser holding compartments comprises three dispenser holding compartments.

15. The case of claim 14, wherein each dispenser holding compartment comprises grippers formed therein.
